(19) United States
(12) Patent Application Publication
(10) Pub. No.: US 2023/0277813 A1
(43) Pub. Date: Sep. 7, 2023

(54) Splitable Tissue Cutting Device

(71) Applicant: Bard Access Systems, Inc., Salt Lake City, UT (US)

(72) Inventor: Glade H. Howell, Draper, UT (US)

(21) Appl. No.: 18/116,249

(22) Filed: Mar. 1, 2023

Related U.S. Application Data
(60) Provisional application No. 63/315,888, filed on Mar. 2, 2022.

(51) Int. Cl. A61M 25/06; A61M 25/09

(52) U.S. Cl. CPC A61M 25/0612; A61M 25/09041

(57) ABSTRACT

Tissue-cutting devices and methods thereof, where a tissue-cutting device is coupleable with a needle used to define a needle tract. The tissue-cutting device includes a blade that cuts the skin during insertion of the device in a needle track to enlarge to needle tract. The tissue-cutting device can be configured for lateral separation from an elongate medical device (e.g., a guidewire). The tissue-cutting device can include a channel for receiving the elongate medical device. The channel can be configured to (i) retain the elongate medical device within the channel in the absence of a deliberate action by the clinician and (ii) facilitate lateral removal of the elongate medical device from the channel in response to the deliberate action. A needle assembly includes a needle coupled with the tissue-cutting device. The tissue-cutting device may include a sheath extending along a distal portion of the tissue-cutting device.

### PRIORITY

This application claims the benefit of priority to U.S. Provisional Application No. 63/315,888, filed Mar. 2, 2022, which is incorporated by reference in its entirety into this application.

### BACKGROUND

Before placing a catheter in a blood vessel of a patient, it is common to nick the patient's skin adjacent a needle tract at an insertion site for dilation of tissue therearound with a dilator. Typically, nicking the patient's skin and dilating the tissue around the needle tract is performed separately. Indeed, the nicking is usually performed with a dedicated skin nicker or a scalpel having a #11 blade; the dilating is usually performed with a dilator two French sizes larger than the catheter being placed. Attempts to integrate the nicking of the patient's skin and the dilating of the tissue around the needle tract have resulted in a spring-loaded blade generally regarded as unsafe due to clinicians not being able to control the blade. What is needed is a tissue-cutting device that safely integrates the nicking of a patient's skin at an insertion site with the dilating of tissue around a needle tract at the insertion site. Such a device would reduce procedural time and errors when, for example, placing a catheter at the insertion site.

Disclosed herein are tissue-cutting devises, assemblies and methods that address the foregoing.

### SUMMARY

Disclosed herein is a tissue-cutting device that, according to some embodiments, includes a body defining a distal end and a proximal end, where the body includes (i) a channel wall defining a channel extending along a length of the body, (ii) one or more protrusions extending laterally away from the channel wall adjacent the proximal end, and (iii) a sheath portion extending proximally away from the distal end, where the sheath portion is disposed in line with the channel. The device further includes a blade attached to the body along a medial portion of the body, where the blade extends laterally away from the channel wall so that a cutting edge of the blade is disposed opposite the channel. The channel is configured to receive an elongate medical device therein, so as to selectively inhibit and allow lateral removal of the elongate medical device from the channel. The cutting edge is disposed at an angle with respect to the channel so that a distal portion of the cutting edge is positioned closer to the channel than a proximal portion of the cutting edge. In some embodiments, the device is slidably displaceable along the elongate medical device. In some embodiments, the sheath portion includes a sheath length between about 1 cm and 2 cm.

The device is configured for insertion along a needle tract extending between a skin surface and a blood vessel wall of a patient. In some embodiments, the one or more protrusions define an insertion stop for the device during insertion of the device along the needle tract and in some embodiments, the insertion stop defines a depth of cut for the blade in accordance the angle of the blade.

In some embodiments, the blade includes a sharp tip at a distal end of the blade, and the sharp tip is disposed between an outside surface of the channel wall and an inside surface of the channel wall and further in some embodiments, a proximal edge of the blade is encapsulated within at least one of the one or more protrusions.

The blade may be formed of a stainless steel and the body may be formed of a thermo-plastic material via an injection molding process. In some embodiments, the blade is insert-molded into the body so that the sharp tip is molded into the channel wall and the back edge is molded into at least one of the one or more protrusions.

In some embodiments, sheath portion includes a taper so that a diameter of the sheath portion adjacent the distal end of the body is less than a diameter of the sheath portion adjacent the blade.

In some embodiments, the channel is configured to allow (i) lateral separation of the device from the elongate medical device in response to the deliberate action by a clinician and (ii) inhibit lateral separation of the device from the elongate medical device in the absence of the deliberate action by the clinician.

In some embodiments, the channel wall defines a gap extending along the channel, where the gap has a width less than a diameter of the elongate medical device. The channel wall is deflectable so as to allow the gap to widen in response to the deliberate action and the deliberate action includes displacing the elongate medical device laterally out of the channel through the gap.

In some embodiments, the channel wall defines a gap extending along the channel, where the gap includes a width less than a diameter of the elongate medical device. The channel wall further includes one or more frangible connecting members extending across the gap and a hinge extending along the channel opposite the gap, where the hinge is disposed between a first channel-wall portion and a second channel-wall portion. The deliberate action includes (i) breaking the one or more frangible connecting members, (ii) widening the gap via rotating the first channel-wall portion with respect to the second channel-wall portion about the hinge and (iii) displacing the elongate medical device laterally out of the channel through the gap. In some embodiments, the hinge is a living hinge formed via the injection molding process.

In some embodiments, the channel wall includes a first longitudinal channel-wall section and a second longitudinal channel-wall section disposed laterally opposite the first longitudinal channel-wall section. The channel wall further includes one or more frangible connecting members attaching the first longitudinal channel-wall section to the second longitudinal channel-wall section. The deliberate action includes breaking the one or more frangible connecting members and separating the first longitudinal channel-wall section from the second longitudinal channel-wall section.

In some embodiments, the elongate medical device is one of a needle or a guidewire. In some embodiments, the elongate medical device is a needle, and the sheath portion includes a sheath length such that a total length of the device is sufficient to extend between a bevel portion of a needle tip and a needle hub. In some embodiments, the body includes a connecting hub disposed at the proximal end of the device, where the connecting hub is configured to couple with the needle hub. In some embodiments, connecting hub includes a Luer taper portion configured to couple with a corresponding Luer taper portion of the needle hub.

Also disclosed herein is a needle assembly, including a needle configured to define the needle tract between a skin surface of a patient and a blood vessel of the patient. The needle assembly further includes any of the tissue-cutting device embodiments summarized above.

In some embodiments of the assembly, the connecting hub of the device is coupled with the needle hub, and in further embodiments, the distal end of the device is disposed adjacent the bevel of the needle.

Also disclosed herein is a method for establishing a blood-vessel access pathway. According to some embodiments, the method includes inserting a needle into a patient from a skin surface to a blood vessel lumen to define a needle tract, where the needle is coupled with a tissue-cutting device and the needle is disposed within a channel extending along a sheath portion of the tissue-cutting device. The method further includes (i) inserting the sheath portion of the tissue-cutting device into the needle tract, (ii) advancing the tissue-cutting device along the needle tract to cause a blade of the tissue-cutting device to cut the skin adjacent the needle tract to enlarge the needle tract, (iii) withdrawing the needle from the needle tract, and (iv) withdrawing the tissue-cutting device from the needle tract.

In some embodiments of the method, advancing the tissue-cutting device includes sliding the tissue cutting device distally along the needle. In some embodiments of the method, the tissue-cutting device is coupled with the needle to define co-movement of the tissue-cutting device and the needle.

In some embodiments, the method further includes inserting the sheath portion into the blood vessel lumen. In some embodiments, the method further includes inserting a guidewire through the needle tract into the blood vessel lumen.

In some embodiments of the method, inserting the guidewire is performed after withdrawing the needle. In some embodiments of the method, advancing the tissue-cutting device is performed after inserting the guidewire. In some embodiments of the method, advancing the tissue-cutting device is performed after withdrawing the needle.

In some embodiments of the method, advancing the tissue-cutting device includes sliding the tissue-cutting device distally along the guidewire, where the guidewire is disposed with the channel.

In some embodiments, the method further includes laterally separating the tissue-cutting device from the guidewire. In some embodiments of the method, separating the tissue-cutting device from the guidewire includes displacing a first portion of the tissue-cutting device with respect to a second portion of the tissue-cutting device to establish a gap along the channel, where the gap has sufficient width to allow lateral passage of the guidewire therethrough.

These and other features of the concepts provided herein will become more apparent to those of skill in the art in view of the accompanying drawings and following description, which describe particular embodiments of such concepts in greater detail.

### DRAWINGS

FIG. 1A illustrates a first embodiment of a tissue-cutting device, in accordance with some embodiments.

FIG. 1B illustrates the tissue-cutting device of FIG. 1A in use with a patient, in accordance with some embodiments.

FIG. 1C is a back view of the tissue-cutting device of FIG. 1A, in accordance with some embodiments.

FIG. 1D is a bottom view of the tissue-cutting device of FIG. 1A, in accordance with some embodiments.

FIG. 2A illustrates a back view of the tissue-cutting device of FIG. 1A having a rotating engagement mechanism disposed in a closed state, in accordance with some embodiments.

FIG. 2B illustrates a back view of the tissue-cutting device of FIG. 1A having the rotating engagement mechanism disposed in an opened state, in accordance with some embodiments.

FIG. 3A illustrates a back view of the tissue-cutting device of FIG. 1A having a separating engagement mechanism disposed in a closed state, in accordance with some embodiments.

FIG. 3B illustrates a back view of the tissue-cutting device of FIG. 1A having the separating engagement mechanism disposed in an opened state, in accordance with some embodiments.

FIG. 4A illustrates a second embodiment of a tissue-cutting device, in accordance with some embodiments.

FIG. 4B illustrates the tissue-cutting device of FIG. 4A in use with a patient, in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or “proximal section” of, for example, a tissue-cutting device includes a portion or section of the tissue-cutting device intended to be near a clinician when the tissue-cutting device is used on a patient. Likewise, a “proximal length” of, for example, the tissue-cutting device includes a length of the tissue-cutting device intended to be near the clinician when the tissue-cutting device is used on the patient. A “proximal end” of, for example, the tissue-cutting device includes an end of the tissue-cutting device intended to be near the clinician when the tissue-cutting device is used on the patient. The proximal portion, the proximal section, or the proximal length of the tissue-cutting device can include the proximal end of the tissue-cutting device; however, the proximal portion, the proximal section, or the proximal length of the tissue-cutting device need not include the proximal end of the tissue-cutting device. That is, unless context suggests otherwise, the proximal portion, the proximal section, or the proximal length of the tissue-cutting device is not a terminal portion or terminal length of the tissue-cutting device.

With respect to “distal,” a “distal portion” or a “distal section” of, for example, a tissue-cutting device includes a portion or section of the tissue-cutting device intended to be near or in a patient when the tissue-cutting device is used on the patient. Likewise, a “distal length” of, for example, the tissue-cutting device includes a length of the tissue-cutting device intended to be near or in the patient when the tissue-cutting device is used on the patient. A “distal end” of, for example, the tissue-cutting device includes an end of the tissue-cutting device intended to be near or in the patient when the tissue-cutting device is used on the patient. The distal portion, the distal section, or the distal length of the tissue-cutting device can include the distal end of the tissue-cutting device; however, the distal portion, the distal section, or the distal length of the tissue-cutting device need not include the distal end of the tissue-cutting device. That is, unless context suggests otherwise, the distal portion, the distal section, or the distal length of the tissue-cutting device is not a terminal portion or terminal length of the tissue-cutting device.

The phrases “connected to,” “coupled to,” and “in communication with” refer to any form of interaction between two or more entities, including but not limited to mechanical, electrical, magnetic, electromagnetic, fluid, and thermal interaction. Two components may be coupled to each other even though they are not in direct contact with each other. For example, two components may be coupled to each other through an intermediate component.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method. Additionally, all embodiments disclosed herein are combinable and/or interchangeable unless stated otherwise or such combination or interchange would be contrary to the stated operability of either embodiment.

References to approximations may be made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially straight” is recited with respect to a feature, it is understood that in further embodiments, the feature can have a precisely straight configuration.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Unless defined otherwise, all technical and scientific terms used herein have the same meaning as commonly understood by those of ordinary skill in the art.

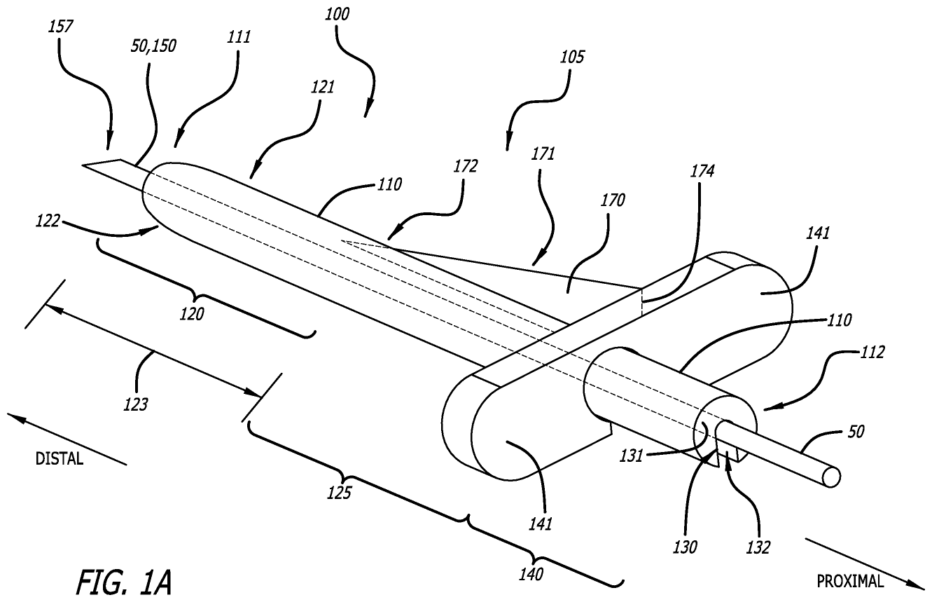

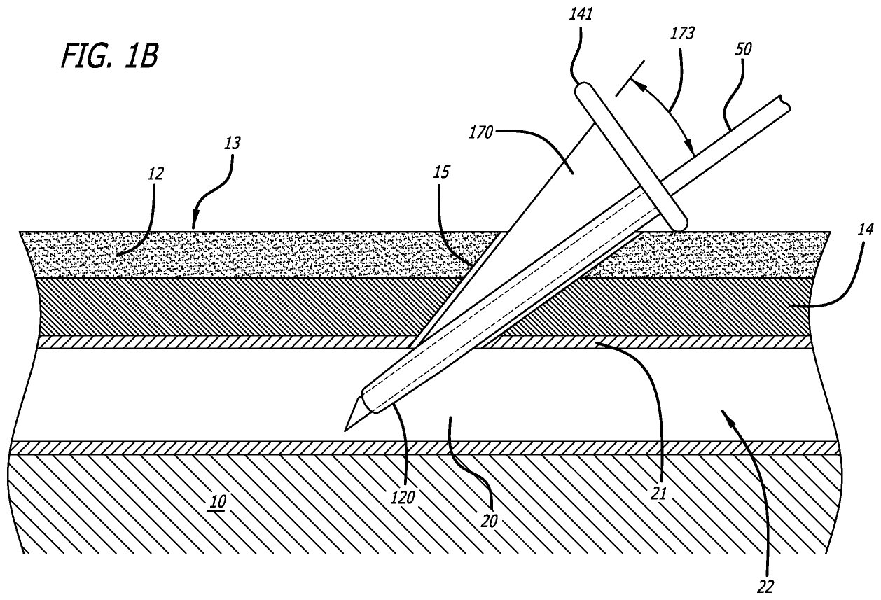

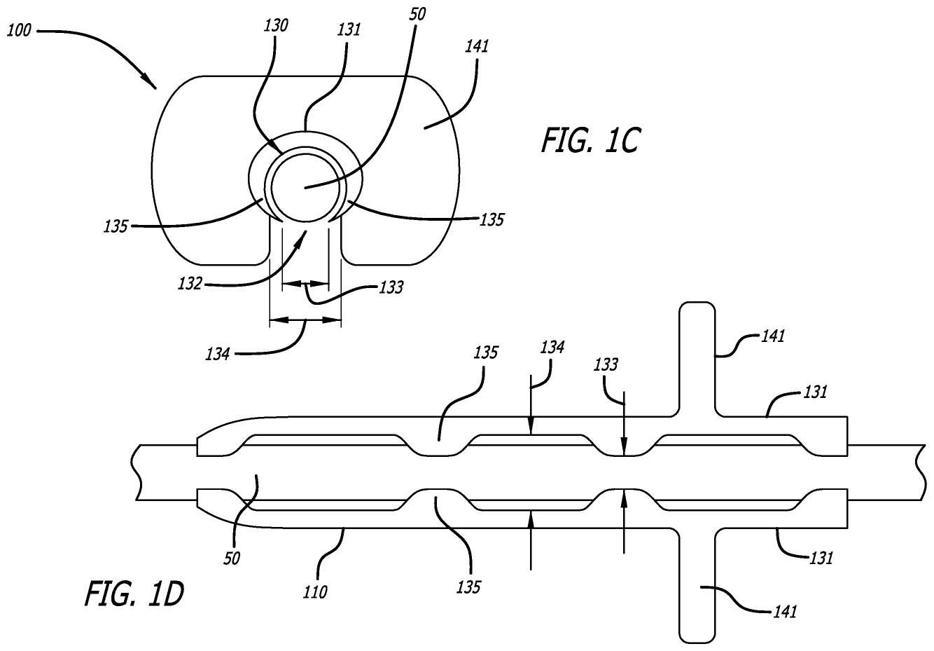

FIGS. 1A-1D illustrate a first embodiment of a tissue-cutting device 100. FIG. 1A is a perspective view of the tissue-cutting device (device) 100 coupled with an elongate medical device 50 (e.g., a needle, a guidewire, or a stylet). FIG. 1B is a side view of the device 100 disposed along a needle tract 15 of a patient 10. The device 100 is generally configured for insertion along the needle tract 15 of a patient 10 extending between a skin surface 13 and a blood vessel wall 21 of the patient 10. The device 100 is also generally configured to enlarge the needle tract 15 by cutting the skin 12 and/or facia 14 surrounding the needle tract 15.

The device 100 generally includes a body 110 having an elongated shape defining a distal end 111 and a proximal end 112. The body 110 includes a channel 130 extending along a length of the body 110 from the distal end 111 and a proximal end 112, and the channel 130 is configured to receive the elongate medical device 50 therein. The channel 130 is formed by a channel wall 131. The channel 130 includes a gap 132 extending along the length of the channel 130. The channel 130 is generally configured to facilitate sliding of the device 100 along a length of the elongate medical device 50. The body 110 may be formed of a thermo-plastic material via an injection molding process.

A tissue-cutting blade 170 is coupled with the body 110 so that a cutting edge 171 extends laterally away from the body 110 along a medial portion 125 of the of the body 110. The blade 170 is generally configured to nick or cut the skin 12 and/or facia 14 in a radially outward direction from the needle tract 15. The cutting edge 171 is disposed at an angle 173 with respect to the channel 130 so that a distal portion of the cutting edge 171 is positioned closer to the channel 130 than a proximal portion of the cutting edge 171. As such a depth of cut by the blade 170 is generally defined by a depth of insertion of the device 100. Although not shown, the body 110 may taper along the medial portion 125 so that during advancement of the medial portion 125 together with the blade 170, the taper causes dilation of the needle tract 15.

The blade 170 may be formed of a stainless steel. In some embodiments, the blade 170 includes a sharp tip 172 at a distal end of the blade 170, and the sharp tip 172 is disposed between an outside surface of the channel wall 131 and an inside surface of the channel wall 131. In some embodiments, the blade 170 is insert molded into the body 110 so that the sharp tip 172 is molded into the channel wall 131.

The body 110 includes one or more protrusions 141 extending laterally away from the channel wall 131 along a handle portion 140 of the body 110 adjacent the proximal end 112. The protrusions 141 may form a handle for the device 100, i.e., a clinician may grip and manipulate the device 100 via the protrusions 141. In the illustrated embodiment, the body 110 includes two protrusions 141 extending away from each other in opposite directions. However, in other embodiments, the body 110 may include 1, 3, 4 or more protrusions 141. The protrusions 141 may take any suitable shape. For example, the protrusions 141 may form a back wall, as illustrated, where the back wall is oriented substantially perpendicular to the body 110. In other embodiments, the protrusions 141 may form a longitudinal wall extending away from the body 110 parallel to the body 110.

In some embodiments, the protrusions 141 or a portion thereof may define an insertion stop for the device 100 during insertion of the device 100 along the needle tract 15, i.e., during insertion, a portion of a protrusion 141 may abut the skin surface 13 and prevent further insertion of the device 100. In some embodiments, the insertion stop may define a depth of cut for the blade 170 in accordance the angle 173 of the blade 170. In some embodiments, a proximal edge 174 of the blade 170 is encapsulated within at least one of the protrusions 141.

A sheath portion 121 of the body 110 is disposed along a distal portion 120 of body 110. The sheath portion 121 is generally configured for insertion through the needle tract 15. The channel 130 extends along the sheath portion 121. In some embodiments, the sheath portion 121 includes a taper 122 adjacent the distal end 111 so that a diameter of the sheath portion 121 adjacent the distal end 111 is less than a diameter of the sheath portion 121 adjacent the blade 170. In some embodiments, the sheath portion 121 includes a sheath length 123 of about 1 cm to 2 cm.

In some embodiments, the elongate medical device 50 may be a needle 150 configured for defining the needle tract 15, and the needle 150 may be combined with the device 100 to define a needle assembly 105. More specifically, the needle assembly 105 includes the device 100 having the needle 150 disposed within the channel 130. In some embodiments, the device 100 may be positioned along the needle 150 so that the distal end 111 is disposed adjacent a bevel 157 of the needle 150.

FIGS. 1C and 1D illustrate a first embodiment of an engagement mechanism generally configured to (i) inhibit lateral separation of the device 100 from the elongate medical device 50 in the absence of a deliberate action by the clinician and (ii) allow lateral separation of the device 100 from the elongate medical device 50 in response to the deliberate action by the clinician. FIG. 1C illustrates a back view of the device 100 coupled with the elongate medical device 50 and the FIG. 1D is a bottom view of the device 100. Referring to the FIGS. 1C and 1D, in the illustrated embodiment, the channel wall 131 defines a gap 132 extending along the channel 130. The gap 132 includes one or more portions having an interference gap width 133. The gap 132 may also include one or more portions having clearance gap width 134. The interference gap width 133 is less than a diameter of the elongate medical device 50 and the clearance gap width 134 is greater than the diameter of the elongate medical device 50.

The channel wall 131 includes one or more deflectable portions 135 extending across the clearance gap width 134 to define the interference gap width 133. The deflectable portions 135 are configured to retain the elongate medical device 50 within the channel 130 during use of the device 130. The deflectable portions 135 are configured to deflect outward toward the clearance gap width 134 to allow the elongate medical device 50 to laterally displace out of the channel 130 through the gap 132 when the clinician pulls or otherwise forces the elongate medical device 50 out of the channel 130 (i.e., performs the deliberate action). In some embodiments, the deflectable portions 135 may extend along the entire length of the device 100.

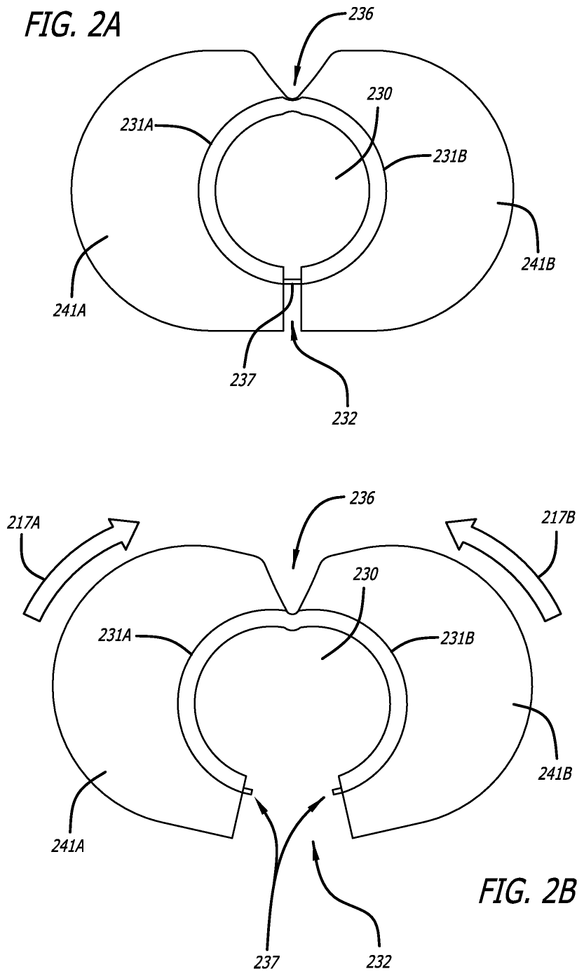

FIGS. 2A-2B illustrate a second embodiment of the engagement mechanism that may be incorporated into the device 100, where the second engagement mechanism facilitates separation of the device 100 from the elongate medical device 50 by rotating a first body portion 231A with respect to a second body portion 231B along a hinge 236. The first body portion 231A is rotatable with respect to the second body portion 231B between a closed state, where the elongate medical device 50 is laterally constrained within the channel 230 and an open state, where the elongate medical device 50 is laterally removeable from the channel 230 through the gap 232. The hinge 236 may be a living hinge formed via an injection molding process.

In some embodiments, one or more frangible connecting members 237 may extend across the gap 232 between the first and second body portions 231A, 231B when the first and second body portions 231A, 231B are disposed in the closed position. The frangible connecting members 237 prevent rotation of first body portion 231A with respect to the second body portion 231B, thereby preventing lateral removal of the elongate medical device 50 from the channel 230, in the absence of the clinician performing the deliberate action. The deliberate action may include applying opposing rotating forces 217A, 217B to the protrusions 241A, 241B, respectively to break the frangible connecting members 237. The clinician may further rotate the first and second body portions 231A, 231B relative to each other to widen that gap 232 sufficiently to facilitate lateral removal of the elongate medical device 50 from the channel 230 through the gap 232.

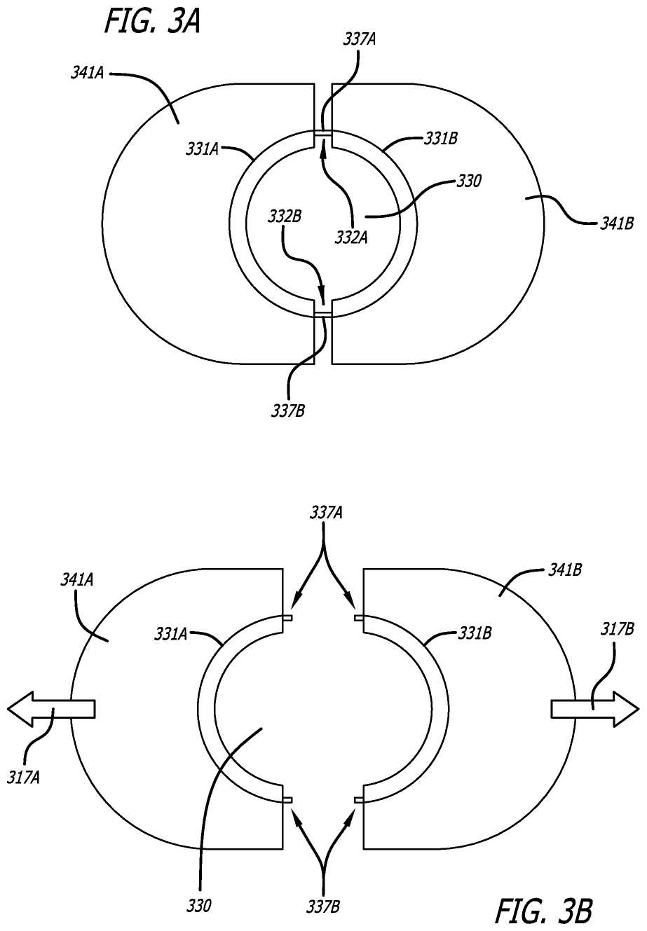

FIGS. 3A-3B illustrate a third embodiment of the engagement mechanism that may be incorporated into the device 100, where the engagement mechanism facilitates separation of the device 100 from the elongate medical device 50 by separating a first channel-wall section 331A from a channel-wall section 331B along gaps 332A, 332B. The first and second channel-wall sections 331A, 331B are attached together via a number of frangible connecting members 337A, 337B extending across gaps 332A, 332B, respectively, defining a closed state of the channel 330. The channel 330 is transitioned to an open state by breaking the frangible connecting members 337A, 337B and separating the first and second channel-wall sections 331A, 331B away from each other.

The frangible connecting members 337A, 337B prevent separation of the first and second channel-wall sections 331A, 331B, thereby preventing lateral removal of the elongate medical device 50 from the channel 332, in the absence of the clinician performing the deliberate action. The deliberate action may include applying opposing forces 317A, 317B to the protrusions 341A, 341B, respectively to break the frangible connecting members 337A, 337B. The clinician may further separate the first and second channel-wall sections 331A, 331B away from each other to facilitate lateral removal of the elongate medical device 50 from the channel 330.

A method for establishing a blood vessel access pathway utilizing the tissue-cutting device 100 may include all or any subset of the following steps or processes, in accordance with some embodiments. The method may be performed by a medical technician in accordance with a vascular access procedure. Also disclosed herein is a method for establishing a blood-vessel access pathway. The clinician may insert a needle into a patient from a skin surface to a blood vessel lumen to define a needle tract, where the needle is coupled with the tissue-cutting device and the needle is disposed within the sheath portion of the tissue-cutting device. The clinician may further insert the sheath portion of the tissue-cutting device into the needle tract. The clinician may further advance the tissue-cutting device along the needle tract to cause the blade of the tissue-cutting device to cut the skin adjacent the needle tract to enlarge the needle tract. After establishing and enlarging the needle tract, the clinician may withdraw the needle and the tissue-cutting device from the needle tract.

Advancement the tissue-cutting device may include sliding the tissue-cutting device distally along the needle. The clinician may further insert a guidewire through the needle tract into the blood vessel lumen. Inserting the guidewire through the needle tract may include inserting the guidewire through the needle.

In some embodiments, the clinician may laterally separate the tissue-cutting device from the guidewire, i.e., without threading the tissue-cutting device off either end of the guidewire. In separating the tissue-cutting device from the guidewire, clinician may displace a first portion of the tissue-cutting device with respect to a second portion of the tissue-cutting device to establish a gap along the channel having sufficient width to allow lateral passage of the guidewire therethrough. The clinician may also laterally separate the tissue-cutting device from the needle via the gap.

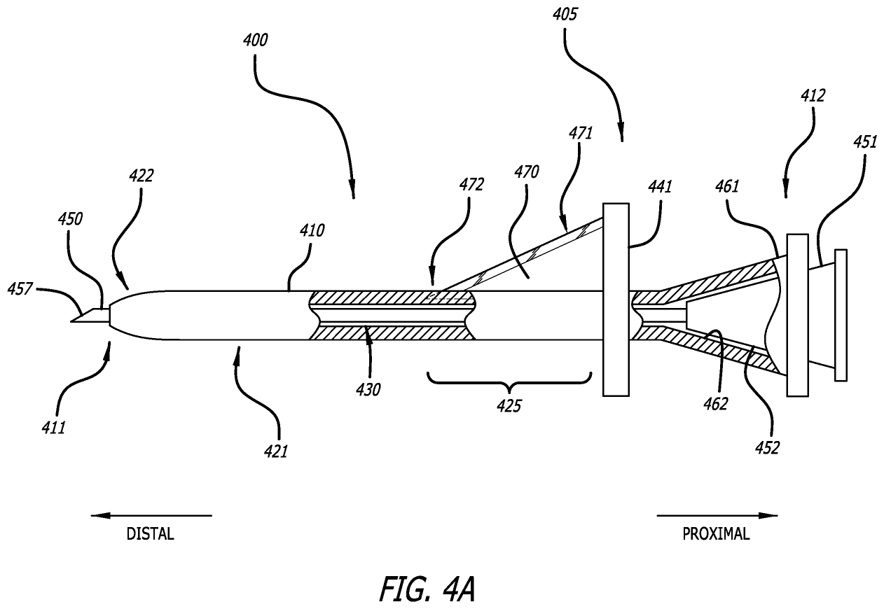

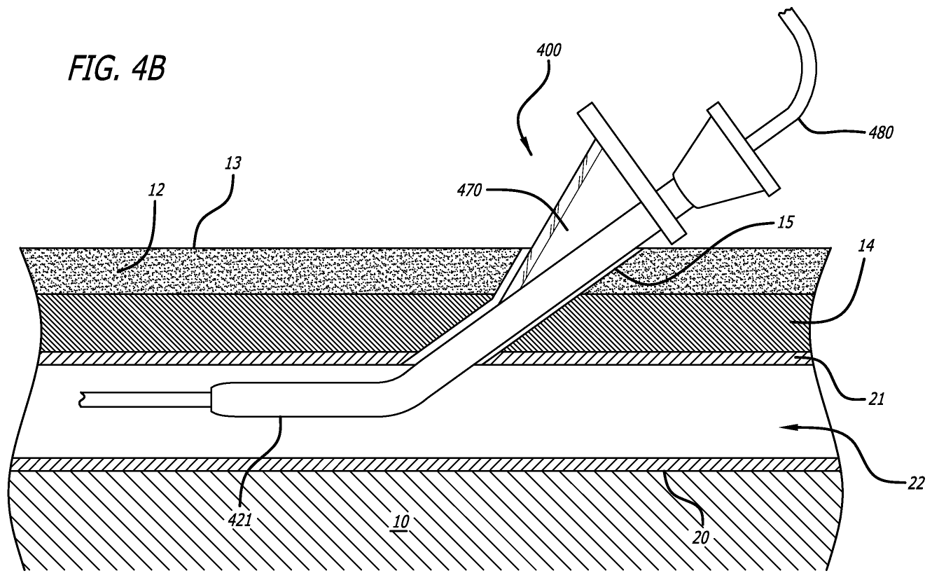

FIGS. 4A-4B illustrate a second embodiment of the tissue-cutting device that can, in certain respects, resemble components of the tissue-cutting device described in connection with FIGS. 1A-3B. It will be appreciated that all the illustrated embodiments may have analogous features. Accordingly, like features are designated with like reference numerals. Relevant disclosure set forth above regarding similarly identified features thus may not be repeated hereafter. Moreover, specific features of the tissue-cutting device and related components shown in FIGS. 1A-3B may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the tissue-cutting device of FIGS. 4A-4B. Any suitable combination of the features, and variations of the same, described with respect to the tissue-cutting device and components illustrated in FIGS. 1A-3B can be employed with the tissue-cutting device and components of FIGS. 4A-4B, and vice versa.

FIG. 4A illustrates a side view of the tissue-cutting device (device) 400 coupled with a needle 450 and FIG. 4B illustrates the device 400 in use with a patient 50. The following description refers to FIGS. 4A and 4B. The device 400 is generally configured for insertion along the needle tract 15 of the patient 10 extending between a skin surface 13 and the blood vessel wall 21 of the patient 10. The device 400 is further configured for advancement along the blood vessel 20 within the blood vessel lumen 22. The device 400 is also generally configured to enlarge the needle tract 15 by cutting the skin 12 and/or facia 14 surrounding the needle tract 15.

The device 400 generally includes a body 410 having an elongated shape defining a distal end 411 and a proximal end 412. The body 410 includes a channel 430 extending along a length of the body 410 from the distal end 411 and a proximal end 412, and the channel 430 is configured to receive the needle 450 therein. The channel 430 is also configured to receive other elongate medical devices therein such as a guidewire 480 as shown in FIG. 4B.

The channel 430 includes an engagement mechanism generally configured to inhibit lateral separation of the device 400 from the needle 450 and/or the guidewire 480 in the absence of the deliberate action by the clinician and allow lateral separation of the device 400 from the elongate medical device 50 in response to the deliberate action by a clinician. In some embodiments, the engagement mechanism may in certain respects resemble the mechanism described above in relation to FIGS. 3A-3B. More specifically, the body 410 may be splitable along the channel 430 in accordance with the deliberate action of clinician. The channel 430 is generally configured to facilitate sliding of the device 400 along needle 450 and/or the guidewire 480.

A tissue-cutting blade 470 is coupled with the body 410 so that a cutting edge 471 extends laterally away from the body 410 along a medial portion 425. The blade 470 is generally configured to nick or cut the skin 12 and/or facia 14 in a radially outward direction from the needle tract 15. The cutting edge 471 is disposed at an angle with respect to the channel 430 so that a distal portion of the cutting edge 471 is positioned closer to the channel 430 than a proximal portion of the cutting edge 471. As such, a depth of cut by the blade 470 may be defined by a depth of insertion of the device 400. The blade 470 may be formed of a stainless steel. In some embodiments, the blade 470 includes a sharp tip 472 at a distal end of the blade 470, and the sharp tip 472 may be disposed below an outside surface of the body 410. Although not shown, the body 410 may include a taper along the medial portion 425 so that during advancement of the medial portion 425 together with the blade 470, the taper causes dilation of the needle tract 15.

The body 410 includes one or more protrusions 441 extending laterally away from the body 410. The protrusions 441 may form a handle for the device 400, i.e., a clinician may grip and manipulate the device 400 via the protrusions 441. In some embodiments, the protrusions 441 or a portion thereof may define an insertion stop for the device 400 during insertion of the device 400 along the needle tract 15, i.e., during insertion a portion of a protrusion 441 may abut the skin surface 13 and prevent further insertion of the device 400. In some embodiments, the insertion stop may define a depth of cut for the blade 470 in accordance the angle of the blade 470.

A sheath portion 421 of the body 410 is disposed along a distal portion of body 410. The sheath portion 421 is generally configured for insertion through the needle tract 15 into the blood vessel lumen 22 as shown in FIG. 4B. In some embodiments, the sheath portion 421 includes a taper 422 adjacent the distal end 411. The sheath portion 421 may include a flexibility to accommodate an angular difference between the needle tract 12 and the blood vessel 20. In other words, the sheath portion 421 may be configured to bend during use so that the sheath portion 421 may be advanced along the blood vessel lumen 422.

In use, the needle 450 may be withdrawn from the device 400, and the guidewire 480 may be inserted through the device 400 (i.e., the channel 430). The device 400 may provide a guide for the guidewire 480 during advancement of the guidewire 480 along the needle tract 15 and into the blood vessel lumen 22.

The device 400 further includes a connecting hub 461 coupled with the body 410 at the proximal end 412. The connecting hub 461 is generally configured to engage a needle hub 451 of the needle 450. More specifically, the connecting hub 461 is configured to selectively attach to the needle hub 451. In some embodiments, the connecting hub 461 may resemble certain features and functionalities of a female Luer connector. Similarly, the needle hub 451 may resemble certain features and functionalities of a corresponding male Luer connector. In some embodiments, the connecting hub 461 may include a female Luer taper 462 for receiving and coupling with a male Luer taper 452 of the needle hub 451.

A length of the sheath portion 421 may correspond to a length of the needle 450 such that, when the connecting hub 461 is coupled with the needle hub 451, the distal end 411 of the device 400 (i.e., the distal end of the sheath portion 421) is disposed adjacent the bevel 457 of the needle 450. As the connecting hub 461 is coupled with the body 410, the connecting hub 461 is also splitable together with the body 410 to allow lateral separation of the connecting hub 461 from the needle 450 and/or the guidewire 480.

In some embodiments, the needle 450 may be combined with the device 400 to define a needle assembly 405. More specifically, the needle assembly 405 includes the device 400 having the needle 450 disposed within the channel 430. In some embodiments, the connecting hub 451 may be coupled with the needle hub 461 so that the distal end 411 is disposed adjacent the bevel 457 of the needle 450.

A method of for establishing a blood vessel access pathway utilizing the tissue-cutting device 400 may include all or any subset of the following steps or processes, in accordance with some embodiments. The method may generally be performed by a medical technician in accordance with a vascular access procedure. Also disclosed herein is a method for establishing a blood-vessel access pathway. The clinician may insert a needle into a patient from a skin surface to a blood vessel lumen to define a needle tract, where the needle is coupled with the tissue-cutting device and the needle is disposed within the sheath portion of the tissue-cutting device. The clinician may further insert the sheath portion of the tissue-cutting device into the needle tract. The clinician may further advance the tissue-cutting device along the needle tract to cause the blade of the tissue-cutting device to cut the skin adjacent the needle tract to enlarge the needle tract. After establishing and enlarging the needle tract, the clinician may withdraw the needle and the tissue-cutting device from the needle tract.

Advancing the tissue-cutting device may include sliding the tissue-cutting device distally with respect to the needle. The clinician may further insert the sheath portion into the blood vessel lumen. The clinician may further insert a guidewire through the needle tract into the blood vessel lumen. The clinician may insert the guidewire after withdrawing the needle. The clinician may advance the tissue-cutting device is performed after inserting the guidewire. The clinician may advance the tissue-cutting device after withdrawing the needle. Advancing the tissue-cutting device may include sliding the tissue-cutting device distally along the guidewire, where the guidewire is disposed with the channel.

During insertion of the needle, the tissue-cutting device may be coupled with the needle to define co-movement of the tissue-cutting device and the needle so that insertion of the sheath portion and insertion of the needle occur simultaneously.

In some embodiments, the clinician may laterally separate the tissue-cutting device from the guidewire, i.e., without threading the tissue-cutting device off either end of the guidewire. In separating the tissue-cutting device from the guidewire, clinician may displace a first portion of the tissue-cutting device with respect to a second portion of the tissue-cutting device to establish a gap along the channel having sufficient width to allow lateral passage of the guidewire therethrough.

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures may be made from the particular embodiments disclosed herein without departing from the scope of the concepts provided herein.

1. A tissue-cutting device, comprising:
a body defining a distal end and a proximal end, the body including:
a channel wall defining a channel extending along a length of the body;
one or more protrusions extending laterally away from the channel wall adjacent the proximal end; and
a sheath portion extending proximally away from the distal end, the sheath portion disposed in line with the channel; and
a blade attached to the body along a medial portion of the body, the blade extending laterally away from the channel wall so that a cutting edge of the blade is disposed opposite the channel, wherein:
the channel is configured to receive an elongate medical device therein, so as to (i) inhibit lateral removal of the elongate medical device from the channel, and (ii) selectively allow lateral removal of the elongate medical device from the channel, and
the cutting edge is disposed at an angle with respect to the channel so that a distal portion of the cutting edge is disposed closer to the channel than a proximal portion of the cutting edge.

2. The device of claim 1, wherein the device is slidably displaceable along the elongate medical device.

3. The device of claim 1, wherein the device is configured for insertion along a needle tract extending between a skin surface and a blood vessel wall of a patient.

4. The device of claim 1, wherein the one or more protrusions provide an insertion stop for the device during insertion of the device along the needle tract.

5. The device of claim 4, wherein the insertion stop defines a depth of cut for the blade in accordance with the angle of the blade.

6. The device of claim 1, wherein:
the blade includes a sharp tip at a distal end of the blade, and
the sharp tip is disposed between an outside surface of the channel wall and an inside surface of the channel wall.

7. The device of claim 1, wherein a proximal edge of the blade is encapsulated within at least one of the one or more protrusions.

8. The device of claim 1, wherein the blade is formed of a stainless steel.

9. The device of claim 1, wherein:
the body is formed of a thermo-plastic material via an injection molding process, and
the blade is insert molded into the body so that the sharp tip is molded into the channel wall and the back edge is molded into the back wall.

10. The device of claim 1, wherein sheath portion includes a taper so that a diameter of the sheath portion adjacent the distal end of the body is less than a diameter of the sheath portion adjacent the blade.

11. The device of claim 1, wherein the channel is configured to:
allow lateral separation of the device from the elongate medical device in response to a deliberate action by a clinician, and
inhibit lateral separation the device from the elongate medical device in the absence of the deliberate action by the clinician.

12. The device of claim 1, wherein:
the channel wall defines a gap extending along the channel, the gap having a width less than a diameter of the elongate medical device;
the channel wall is deflectable so as to allow the gap to widen in response to the deliberate action; and
the deliberate action includes displacing the elongate medical device laterally out of the channel through the gap.

13. The device of claim 1, wherein:
the channel wall defines a gap extending along the channel, the gap having a width less than a diameter of the elongate medical device and one or more frangible connecting members extending across the gap;
the channel wall further defines a hinge extending along the channel opposite the gap, the hinge disposed between a first channel-wall portion and a second channel-wall portion; and
the deliberate action includes:
breaking the one or more frangible connecting members,
widening the gap via rotating the first channel-wall portion with respect to the second channel-wall portion about the hinge; and
displacing the elongate medical device laterally out of the channel through the gap.

14. The device of claim 13, wherein the hinge is a living hinge is formed via the injection molding process.

15. The device of claim 1, wherein:
the channel wall includes:
a first longitudinal channel-wall section;
a second longitudinal channel-wall section disposed laterally opposite the first longitudinal channel-wall section; and
one or more frangible connecting members attaching the first longitudinal channel-wall section to the second longitudinal channel-wall section; and
the deliberate action includes:
breaking the one or more frangible connecting members; and
separating the first longitudinal channel-wall section from the second longitudinal channel-wall section.

16. The device of claim 1, wherein the sheath portion defines a length between about 1 cm and 2 cm.

17. The device of claim 1, wherein the elongate medical device is one of a needle or a guidewire.

18. The device of claim 1, wherein;
the elongate medical device is a needle, and
the sheath portion includes a sheath length such that a total length of the device is sufficient to extend between a bevel portion of a needle tip and a needle hub.

19. The device of claim 18, wherein the body includes a connecting hub disposed at the proximal end of the device, the connecting hub configured to couple with the needle hub.

20. The device of claim 19, wherein the connecting hub includes a Luer taper portion configured to couple with a corresponding Luer taper portion of the needle hub.

21. A needle assembly, comprising:
a needle configured to define needle tract between a skin surface of a patient and a blood vessel of the patient; and
the tissue-cutting device of any of claims 1-20 disposed over a shaft of the needle.

22. The assembly of claim 21, wherein a connecting hub of the device is coupled with a needle hub.

23. The assembly of claim 22, wherein the distal end of the device is disposed adjacent the bevel of the needle.

24-34. (canceled)
